(19) United States
(12) Patent Application Publication
(10) Pub. No.: US 2004/0214276 A1
(43) Pub. Date: Oct. 28, 2004

(54) Production of glycosylated macrolides in E. coli

(76) Inventors: Chaitan Khosla, Palo Alto, CA (US); Hugo Gramajo, Berkeley, CA (US); Kinya Hotta, Pasadena, CA (US); Seiji Kobayashi, Sagamihara (JP)

Correspondence Address: MORRISON & FOERSTER LLP, 3811 VALLEY CENTRE DRIVE, SUITE 500, SAN DIEGO, CA, 92130-2332, US

(21) Appl. No.: 10/632,682

(22) Filed: Jul. 31, 2003

Related U.S. Application Data
(60) Provisional application No. 60/400,122, filed on Jul. 31, 2002.

(51) Int. Cl. C12N 9/00; C12N 9/10; C07H 21/04; C12N 1/21

(52) U.S. Cl. 435/69.1; 435/183; 435/252.33; 435/193; 435/320.1; 536/23.2

(57) ABSTRACT

Methods and materials are provided for E. coli host cells containing an expression system for producing NDP 6-deoxy-sugar, which may also comprise an expression system for expressing 6-deoxyglycosyl transferase and/or an expression system for producing a polyketide and/or polyketide oxidation enzymes.

### CROSS-REFERENCE TO RELATED APPLICATIONS

This application claims priority under 35 U.S.C. §119(e) to U.S. provisional application Serial No. 60/400,122, filed 31 Jul. 2002, which is incorporated herein by reference in its entirety.

### STATEMENT OF RIGHTS TO INVENTIONS MADE UNDER FEDERALLY SPONSORED RESEARCH

This invention was made with U.S. government support from the National Institutes of Health (NIH-R01CA66736). The U.S. government may have certain rights in this invention.

### FIELD OF THE INVENTION

The invention relates to methods and materials relating to a recombinant Escherichia coli (E. coli) host cell containing an expression system for producing a nucleotide diphosphate 6-deoxy-sugar. The host cell may also comprise an expression system for producing a 6-deoxyglycosyl transferase, and an expression system for producing a polyketide to produce a glycosylated polyketide. More specifically, the invention relates to an E. coli host cell containing one or more an expression systems for producing erythromycins or intermediates thereto.

### BACKGROUND OF THE INVENTION

Polyketides (PK's) are a class of natural products with many useful clinical and agricultural applications. In recent years, diversification of the core carbon framework of PK's (aglycones) has been achieved through rational and combinatorial protein engineering approaches in an attempt to expand the scope of biological and chemical properties of PK's. However, in some cases the non-aglycone portion of PK's also plays a critical role in determining the properties of the PK's. For instance, the widely-used antibiotic erythromycin A (1) requires the presence of two deoxysugar moieties, L-cladinose and D-desosamine, for it to exhibit its full antibacterial potency; the corresponding aglycone, 6-deoxyerythronolide B (6-dEB 3), shows no antibacterial activity. Therefore, a convenient technique that allows modification of various aglycones with deoxysugars should be of great value to exploring the novel biological activities of natural as well as artificial PK's. The ability to produce these modified polyketides in industrially-friendly organisms such as Escherichia coli would also be of great value.

### SUMMARY OF THE INVENTION

The invention relates to a recombinant E. coli host cell containing an expression system for producing a nucleotide diphosphate 6-deoxy-sugar. In a preferred embodiment, the sugar may be selected from the group consisting of desosamine, cladinose, mycaminose, oleandrose, forosamine, daunosamine, mycarose, ascarylose, rhamnose, and mycosamine and most preferably, the sugar is D-desosamine or mycarose, and more preferably both. These sugars may be produced using biosynthesis genes from organisms such as Streptomyces venezuelae, Saccharopolyspora erythraea, Streptomyces narbonensis, Streptomyces antibioticus, Streptomyces fradiae, Yersinia pseudotuberculosis, Salmonella enterica, Streptomyces noursei or Streptomyces nodosus. In a preferred embodiment, desosamine may be produced using biosynthesis genes from organisms such as Streptomyces venezuelae, Saccharopolyspora erythraea, Streptomyces antibioticus, or Streptomyces narbonensis, most preferably, the desosamine biosynthesis genes are from Streptomyces venezuelae or S. narbonensis. In a preferred embodiment, the desosamine biosynthesis genes comprise des1-des VI and des VIII genes from Streptomyces venezuelae.

The expression system may further comprise a gene for expressing a 6-deoxyglycosyl transferase such as desosaminyltransferase or mycarosyltransferase. In a preferred embodiment, the 6-deoxyglycosyl transferase expression system comprises a desosaminyltransferase gene such as the des VII gene from Streptomyces venezuelae.

The expression system may also further comprise an expression system for the synthesis and modification of a polyketide which may comprise genes encoding a 6-deoxyerthronolide B synthase, 6-deoxyerythronolide B 6-hydroxylase, erythromycin D 12-hydroxylase and/or erythromycin C 3″-O-methyltransferase, which may be cultured to produce a 6-deoxyerythronolide B. In certain embodiments of the invention, the host cells are modified by introduction of an expression system that provides resistance to macrolide antibiotics. In a one embodiment, the host cells are modified by introduction of an expression system comprising one or more genes encoding erm ribosomal methyltransferases.

The invention is also directed to methods for producing a glycosylated polyketide comprising feeding a polyketide to a culture of the host cells under conditions wherein the nucleotide diphosphate 6-deoxy-sugar is produced, which may further include producing, the 6-deoxyglycosyltransferase, wherein the polyketide is preferably 6-deoxyerythronolide B (which includes analogs thereof).

### BRIEF DESCRIPTION OF THE FIGURES

FIG. 1 represents 4-20% SDS-PAGE of Ni-NTA-column fractions of individually expressed Des enzymes. The numbers to the left indicate the molecular weight of the marker proteins in kilodaltons. M: molecular weight marker; P: flow-through; W: column wash; E: eluate. Expected molecular weight for each of the enzyme is: DesI, 44 kDa; DesII, 53 kDa; DesIII, 30 kDa; DesIV, 36 kDa; DesV, 41 kDa; DesVI, 26 kDa; DesVII, 46 kDa; DesVIII, 42 kDa.

FIG. 2 represents 4-20% SDS-PAGE of Ni-NTA-column fractions of co-expressed eight Des enzymes. M: molecular weight marker; 1: cleared cell lysate; 2: flow-through; 3: column wash; 4: eluate. The numbers on the left side indicate the molecular weight of the marker proteins in kilodaltons.

### DETAILED DESCRIPTION OF THE INVENTION

The invention relates to a recombinant E. coli host cell containing an expression system for producing a nucleotide diphosphate 6-deoxy-sugar. Nucleotide diphosphate sugars are known in the art and may comprise, for example, thymidine-, cytosine- or uracil-diphosphate 6-deoxy-sugar, for example TDP-mycarose or TDP-desosamine.

The preferred sugars are those that are found in glycosylated polyketides. In a preferred embodiment, the sugar may be selected from at least one member of the group consisting of desosamine, cladinose, mycaminose, oleandrose, forosamine, daunosamine, mycarose, ascarylose, rhamnose, and mycosamine and most preferably, the sugar is D-desosamine and/or mycarose.

Desosamine biosynthesis and transfer genes are described with respect to various polyketides and organisms such as erythromycin from Saccharopolyspora erythraea (eryC) in PCT publication WO 97/23630, pikromycin from Streptomyces venezuelae (pikC) in U.S. Pat. No. 6,509,455, oleandomycin from Streptomyces antibioticus in Aguirrezbalaga, infra, (oleG1) and narbomycin from Streptomyces narbonensis in U.S. Pat. No. 6,303,767. Mycaminose biosynthesis and transfer genes related to tylosin from Streptomyces fradiae include tyla, tylB, tylM1, tylM2, and tylM3. Mycarose biosynthesis and transfer genes are described with respect to erythromycin from Saccharopolyspora erythraea (eryB) in PCT Publication WO 97/23630. Oleandrose and olivose biosynthesis and transfer genes as described with respect to oleandomycin in Streptomyces antibioticus, Aguirrezbalaga, infra.

The desosamine may be produced from biosynthesis genes such as from Streptomyces venezuelae, Saccharopolyspora erythraea, Streptomyces antibioticus or Streptomyces narbonensis, most preferably, the desosamine biosynthesis genes are from Streptomyces venezuelae. In a particularly preferred embodiment, the desosamine biosynthesis genes comprises desI-des VI and des VIII genes from Streptomyces venezuelae which natively produces pikromycin. The gene sequence of this desosamine expression system is disclosed in U.S. Pat. No. 6,117,659 which is in incorporated herein by reference in its entirety. Cosmid pKOS023-26, which contains the biosynthetic and transferase genes for producing desosamine, was deposited with the American Type Culture Collection on 20 Aug. 1998 under the Budapest Treaty and is available under the accession number ATCC 203141.

S. narbonensis natively produces desosamine to form, for example, narbomycin. The narbomycin gene cluster is described in U.S. Pat. No. 6,303,767. In a similar fashion to the genes from S. venezulae, the genes involved in desosamine biosynthesis in S. narbonensis are desI-desVI and desVIII, while the desVII gene is a desosaminyltransferase. These genes are highly homologous to those found in S. venezulae.

In addition to S. venezuelae and Streptomyces narbonensis, other organisms natively produce desosamine. For example, Saccharopolyspora erythraea, contains genes that are homologous to the desosamine genes from S. venezuelae and thus are expected to be expressed similarly in E. coli. Similarly, desosamine biosynthesis genes from Streptomyces antibioticus are likewise homologous as described below in more detail.

Specifically, eryCII is a homologue of picCII gene, also known as desVIII, and is believed to encode a 4-keto-6-deoxyglucose isomerase.

eryCVI is a homologue of the picCVI gene, also known as desVI, which encodes a 3-amino dimethyltransferase.

eryCI is a homologue of the picCI gene, also known as des V. It has also been reported that the OleN2 protein, produced from the Streptomyces antibioticus oleandomycin gene cluster, and EryCI are homologues. Please see, Aguirrezbalaga, I., et al., 44 Antimicrobial Agents and Chemotherapy, No. 5, 1266-75 (2000).

eryCVis a homologue of the picCV gene, also known as desII, and is required for desosamine biosynthesis. Aguirrezbalaga also reports that oleT from the Streptomyces antibioticus oleandomycin gene cluster encodes for a protein homologous to DesII from the methymycin and pikromycin pathways and to eryCV, which protein may be 3,4-reductases. Further, Butler, A. et al., Nature Biotechnology, 20, 713-16 (2002) reports homology between NbmJ, expressed from the narbomycin-biosynthetic gene cluster of S. narbonensis, and eryCV.

eryCIVis a homologue of the picCIV gene also known as desI, and is believed to be a 3,4-dehydratase. Aguirrezbalaga also reports that oleNI gene that codes for the OleNI protein, from the Streptomyces antibioticus oleandomycin gene cluster, and eryCIV proteins are homologous. In addition, Butler, et al., supra, reports homology between NbmK, expressed from the narbomycin-biosynthetic gene cluster of S. narbonesis, and eryCIV.

desIV, has no known ery gene homologue and encodes an NDP glucose 4,6-dehydratase. It is believed to be represented by the gdh gene in Sac. erythraea, which lies outside the erythromycin biosynthesis gene cluster. NDP-glucose 4,6-dehydratase is generally used in the production of many different NDP-6-deoxysugars, and as such one gene may serve multiple biosynthetic pathways.

desIII, has no known ery gene homologue and encodes an NDP glucose synthase. It is believed that the homolog of this gene in Sac. erythraea is located outside the erythromycin biosynthesis gene cluster. NDP-glucose synthase is a ubiquitous intermediate in sugar biosynthesis, and as such one gene may serve multiple biosynthetic pathways.

The oleS and oleE genes from the Streptomyces antibioticus oleandomycin gene cluster, involved in desosamine biosynthesis, have been reported, which are involved in both desosamine and oleandrose biosynthesis. Please see, Aguirrezbalaga, J., supra. Regarding oleS, similarities have been reported among dTDP-D-glucose synthases from streptomycetes, such as with MtmD from the mithramycin pathway in Streptomyces argillaceus, StrD from the streptomycin pathway in S. griseus, and DnmL from the daunorubicin pathway in S. peucetius. Mithramycin contains the sugars D-mycarose and D-olivose, whereas daunorubicin contains the aminosugar L-daunosamine.

Aside from TDP-glucose synthase, which may also be produced from Streptomyces fradiae (tylA1) (Merson-Davies & Cundliffe (1994)), there are other genes with functions common to different sugar-biosynthesis pathways, although likewise not necessarily associated with other biosynthesis genes as they often serve many pathways in the cell. For example, TDP-glucose dehydratase is natively produced in Streptomyces fradiae (tylA2) (Merson-Davies & Cundliffe (1994)) or Saccharopolyspora erythraea (gdh) (Linton et al., Gene 1995 Feb. 3; 153(1):33-40). In addition TDP-4-keto-6-deoxyglucose 3,5-epimerase is natively produced in Saccharopolyspora erythraea (kde) (Linton et al., Gene, 1995 Feb. 3; 153(1):33-40). Further, a C5-epimerase is natively produced in Saccharopolyspora erythraea (eryB7) (WO 97/23630) which is only used in making L-configuration sugars.

Based on such homology and commonality of function, similar desosamine biosynthesis pathway genes, including the genes having functions common to different pathways are expected to be similarly expressed and used in E. coli. Specifically, the biosyntheses of the NDP-6-deoxysugars commonly found in polyketide natural products share many common features. From the experimental feeding of labeled precursors, it is known that all ultimately derive from the common primary metabolite D-glucose-1-phosphate. Several of the early steps are common to the known pathways as well. For example, glucose-1-phosphate is first transformed into NDP-glucose by the enzyme NDP-D-glucose synthase, followed by dehydration at C-4 and C-6 by the enzyme NDP-D-glucose 4,6-dehydratase. The resulting intermediate, NDP-4-keto-6-deoxy-D-glucose, serves as a common precursor to the known NDP-6-deoxysugars.

For the aminosugars such as D-desosamine and D-mycaminose, NDP-4-keto-6-deoxy-D-glucose is first converted into the 3-ketosugar through the action of NDP-4-keto-6-deoxy-D-glucose isomerase, and the 3-ketosugar is converted into the NDP-3-amino-6-deoxy-D-glucose by a 3-aminotransferase. For the synthesis of NDP-D-mycaminose, all that remains is N,N-dimethylation via a 3-N-methyltransferase. For the synthesis of NDP-D-desosamine, the 4-position is deoxygenated via a 3,4-dehydratase and a 3,4-reductase prior to the N,N-dimethylation step.

For the L-series 2,6-dideoxysugars, such as L-mycarose, L-oleandrose, and L-cladinose, the NDP-4-keto-6-deoxy-D-glucose is converted into the L-series sugar through the action of a 3,5-epimerase. The 2-hydroxyl is then removed through the action of a 2,3-dehydratase and a 2,3-reductase, analogous to the removal of the 4-hydroxyl in the biosynthesis of D-desosamine described above.

This commonality of precursor and intermediates and similarity in enzymatic transformations suggests that genetic methods demonstrated to be successful for the biosynthesis of a particular NDP-6-deoxysugar in a particular heterologous host can be extended to the biosynthesis of other NDP-6-deoxysugars in that same host.

As such, the host cell is expected to produce 6-deoxy-sugars (other than desosamine) using 6-deoxy-sugar biosynthesis genes from various organisms such as Streptomyces fradiae, Yersinia pseudotuberculosis, Salmonella enterica, Streptomyces noursei or Streptomyces nodosus. In one instance Aguirrezbalaga, supra, reports that the Tylb protein from the tylosin biosynthesis pathway as well as DnrJ from the daunorubicin biosynthesis pathway of Streptomyces peucetius, and LmbS from the lincomycin biosynthesis pathway of Streptomyces lincolnensis, are homologues of eryCIV. The mycarose biosynthesis genes of S. fradiae which produces tylosin as described in Bate, N. et al., Microbiology, 146, 139-46 (2000).

The expression system may further comprise a gene for expressing 6-deoxyglycosyl transferase. Sequence alignments illustrating conserved motifs that correspond to particular folds of glycosyltransferases, which provide strong structural similarities among glycosyltransferases, have been reported, and thus it is expected that a wide range of glycosyltransferases may be used in accordance with the invention. See, e.g., Hu, Y., et al., Chem. and Biol, 9:1287-96 (2002). Examples of genes encoding 6-deoxyglycosyl transferase include mycaminosyl transferase gene from S. fradiae (tylM2), mycarosyl transferase from S. erythraea (eryB5), and the Streptomyces antibioticus olivosyl transferase (oleG2). In a preferred embodiment, the desosaminyl transferase gene and gene product may be from the pikromycin gene cluster (des VII) described herein or may be from a different gene cluster, for example, the desosaminyl transferase gene and gene product from erythromycin (e.g., eryC3), oleandomycin (e.g., oleG1), narbomycin (e.g., des VII) gene clusters as described in WO 97/23630, Aguirrezbalaga, supra, U.S. Pat. No. 6,303,767. Preferably, however, the 6-deoxyglycosyl transferase is not produced from genes from M. megalomicea.

The host cell may also further comprise an expression system for the synthesis of a polyketide, preferably a 6-deoxyerythronolide B (6-dEB). Preferably, the polyketide expression system is not from M. megalomicia. By “a 6-deoxyerythronolide B” is meant a polyketide produced by a 6-deoxy-erythronolide B synthase or variant or mutagenized form thereof. Such variants or mutants may produce analogs of 6-deoxyerythronolide B having altered patterns of alkyl substitution and/or altered degrees of oxidation as described, for example, in U.S. Pat. Nos. 6,403,775; 6,399,789; 6,391,594; and 6,558,942, and PCT Publication WO 03/014312, and they may produce analogs of 6-deoxyerythronolide B having different substituents in place of the 13-ethyl group as described in U.S. Pat. Nos. 6,066,721; 6,500,960; and 6,492,562, and PCT Publication WO 01/31049. For example, a 6-deoxyerythronolide B is intended to include such analogs as 13-methyl-6-deoxyerthronolide B (13-methyl-d-dEB), 11-deoxy-6-deoxyerythronolide B, 8-desmethyl-6-deoxyerythronolide B, 15-fluoro-6-deoxyerythronolide B, 13-propyl-6-deoxyerythronolide B (13-propyl-6-dEB), and similar compounds.

The host cell thus can contain one or more genes that encode enzymes involved in the synthesis and modification of 6-deoxyerthronolide B, for example, 6-deoxyerthronolide B synthase, 6-deoxyerthronolide B 6-hydroxylase, erythromycin D 12 hydroxylase, erythromycin C 3″-O-methyltransferase, or preferably all of the genes that encode the above enzymes.

The biosynthetic pathway for formation of erythromycins begins with the production of the polyketide, 6-deoxyerythronolide B (6-dEB), by the polyketide synthase (6-deoxyerythronolide B synthase, DEBS). In the erythromycin producing organism Saccharopolyspora erythraea, DEBS is encoded by the eryA genes. Homologs of the eryA genes are found, for example, in Streptomyces venezuelae and S. narbonensis. In the next step, 6-dEB is hydroxylated at C-6 to produce erythronolide B through the action of the C-6 hydroxylase, encoded by the eryF gene of Sac. erythraea and its homologs in other organisms. This step is optional, as demonstrated by the production of 6-deoxyerythromycins in strains having an inactivated eryF gene. Subsequently, L-mycarose is attached to the 3-hydroxyl group through the action of the eryBV gene or its homologs from other organisms, using the nucleotide sugar TDP-L-mycarose that is prepared by enzymes encoded by the remaining eryB genes or their homologs from other organisms. The second sugar, D-desosamine, is attached to the 3-O-α-mycarosyl-erythronolide B so produced through the action of the eryCIII desosaminyltransferase and its homologs from other organisms, to produce erythromycin D. Erythromycin D is hydroxylated at C-12 through the action of the C-12 hydroxylase encoded by eryK or its homologs from other organisms, to produce erythromycin C. In the final step, a 3″-O-methyltransferase, encoded by eryG or its homologs from other organisms, adds a methyl group to the mycarosyl unit to covert it to cladinose, thus producing erythromycin A. In the absence of sufficient C-12 hydroxylase activity, the product of the 3″-O-methyl-transferase is the 12-deoxy compound, erythromycin B.

As erythromycins target prokaryotic ribosomes, interfering with protein translation and ultimately resulting in cell death, erythromycin producing organisms must have suitable mechanisms of resistance to the erythromycins they produce. Typically, N6-methylation of a critical adenosine residue (A2058 in E. coli) is sufficient to provide protection for the producing cell, although other mechanisms such as efflux and esterases are available. Host cells of the invention that produce erythromycins thus comprise an expression system for producing a ribosomal methyltransferase capable of methylating A2058 and thus providing protection for the host cell. In one embodiment, the ribosomal methyltransferase is a constitutively expressed member of the erm family of resistance genes, for example the erm gene of Saccharopolyspora erythraea or the ermSF gene of Streptomyces fradiae.

In a more preferred embodiment of the invention, the host cell contains the above enzymes as well as genes encoding enzymes that produce TDP mycarose and mycarosyltransferase, and even more preferably also include an expression system for producing TDP-desosamine and desosaminyltransferase.

The invention is also directed to a method for producing an erythromycin analog comprising culturing the host cells that also contain an expression system for producing both sugars and a polyketide under conditions wherein the erythromycin analog is produced. Examples of such conditions are provided in the Examples below.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLE 1

### Individual Expression of Soluble desI Through desVIII Genes in E. coli

Desosamine has been shown to be essential for the biological activity of erythromycin A (1), (see structure below) and thus the desosaminylation pathway was chosen as the target. Since E. coli is the host organism of choice for its cost and convenience in bioengineering effort, reconstitution of the desosamine biosynthetic pathway from Streptomyces venezuelae (S. venezuelae) in E. coli was undertaken.

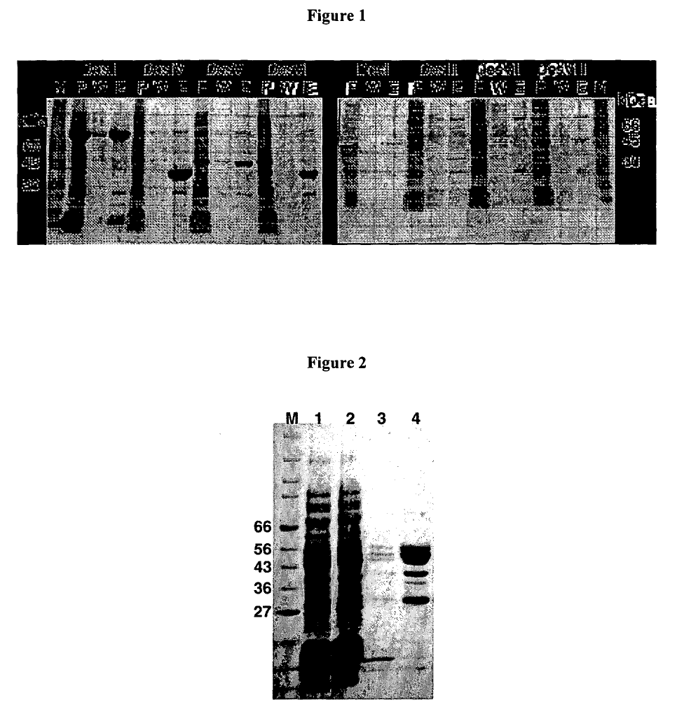

Previously, eight genes have been identified as being involved in the biosynthesis of TDP-desosamine and transfer of this deoxysugar onto aglycones (Xue, et al., PNAS 95, 12111, 1998; U.S. Appl. No. 09/793,708 filed Feb. 22, 2001 (Attorney Docket No. 30062-20021.00), which are incorporated herein by reference). A cosmid clone containing these eight genes from S. venezuelae was obtained from Kosan Biosciences. Each of the eight des genes, that is, desI, desII, desIII, desIV, desV, desVI, desVII, or desVIII, was initially sub-cloned into pET28ac (Novagen, Madison, Wis.) to test whether these proteins can be expressed as soluble proteins in E. coli strain BL21. Cultures were grown in standard Luria-Bertani medium with 50 μg/ml ampicillin at 37° C., 230 rpm until O.D.₆₀₀ reached 0.6. Expression of each target gene was induced by supplementing the culture with isopropyl thiogalactoside (IPTG) to the final concentration of 100 μM. For DesI, II, IV, V, and VI production, culture was incubated at 30° C. for another 6 hours before the cells were harvested by centrifugation. For DesIV, VII, and VIII, culture was incubated at 15° C. for 20 hours. Cell lysates were prepared by sonication on ice, and insoluble materials were removed by centrifugation. The presence of a hexa-his-tag on each of the target enzymes allowed simple enrichment for the enzymes using the nickel-nitrilotriacetic acid (Ni-NTA) metal affinity chromatography (QIAGEN, Valencia, Calif.). Recommended purification protocol was followed. All genes were expressed in soluble form, although variation in the expression level among different enzymes existed (FIG. 1). All proteins migrated at the expected molecular weight. This result indicated that the S. venezuelae des genes could be expressed in E. coli in a soluble form.

### EXAMPLE 2

### Co-Expression of All Eight des Genes in E. coli

Encouraged by the above results, all eight des genes were assembled into a single pET28 construct (pKH26). In this construct, all genes were under the control of a single lac promoter with each gene flanked by a ribosome binding site along with a hexa-his-tag at their 5′ ends. The same culture condition was used for the des gene expression from this multi-cistronic construct except that the culture was incubated at 15° C. for 20 hours after induction. Lysate preparation and protein enrichment was performed as described above. Similarity of molecular weight among the eight Des proteins made it difficult to determine with certainty whether all eight genes were being expressed. However, SDS-PAGE on the Ni-NTA column eluate indicated that all genes were likely to be expressed in soluble form (FIG. 2).

### EXAMPLE 3

### Bioconversion of 13-methyl-, 13-ethyl-, and 13-propyl-6-dEB into desosaminylated products

To verify that all eight des genes are not only expressed but are also metabolically active, an in vivo feeding experiment was performed. In this experiment E. coli BL21 transformed with pKH26 was grown under the same condition as for the protein production experiment except that 1) various 6-deoxyerythronolide B aglycones were fed to the culture and that 2) the induced culture was grown at 18° C. for 24 hours. The supernatant of the culture was extracted with three volumes of ethyl acetate/triethylamine (99:1). The extract was evaporated to dryness and dissolved in a small volume of methanol for analysis with liquid chromatography/mass spectroscopy (LC/MS) for the presence of the desosaminylated aglycones. As a control for validating the effectiveness of the product extraction scheme, the LC/MS analysis of the extract from the culture supplemented with an authentic sample of erythromycin A (1) (Sigma, St. Louis, Mo.) was performed. A mass peak corresponding to that of 1 was clearly identified (Table 1). Next, the extract from the culture with a mixture of 13-methyl-(2), 13-ethyl-(3), and 13-propyl-6-dEB (4) was analyzed. Compounds 2 and 3 were produced in-house, whereas compound 4 was a gift from Kosan Biosciences. In each case mass peaks corresponding to the molecular weight of the expected 5-desosaminylated product of the corresponding aglycone were present. In contrast, the extract from the culture where only 4 was fed exhibited only the mass peak corresponding to 5-desosaminyl-13-propyl-6-dEB, providing further support that the observed peaks are those corresponding to the desosaminylated aglycones. From the culture where no aglycone was fed, none of the desosaminylated aglycone peaks were observed.

### Chemical Structures of the Compounds Discussed in Examples

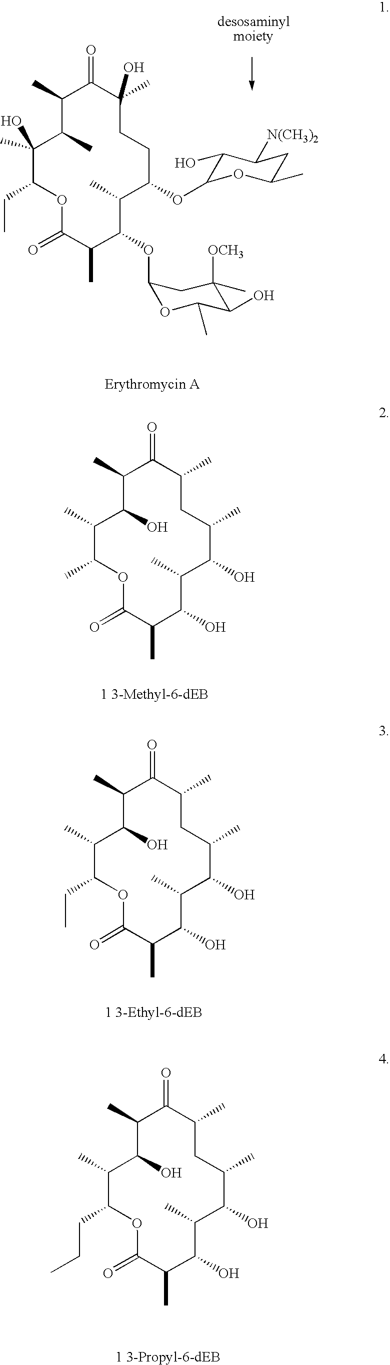 TABLE 1
Comparison of the molecular weight expected of the desosaminylated
aglycones and that observed in the LC/MS analysis of the extract
from E. coli culture fed with (a) a mixture of 13-methyl-, 13-ethyl-.
and 13-propyl-6-dEB, and (b) only 13-propyl-6-dEB.

| | | Expected | |
|---|---|---|---|
| | | molecular weight | Observed molecular weight |
| | Aglycone(s) fed | (MH⁺, daltons) | (MH⁺, daltons) |
| | 13-methyl-6-dEB | 530.72 | 530.89 |
| (a) | 13-ethyl-6-dEB | 544.74 | 544.94 |
| | 13-propyl-6-dEB | 558.77 | 558.94 |
| (b) | 13-propyl-6-dEB | 558.77 | 558.79 |

The above experiments have shown that E. coli can successfully synthesize TDP-desosamine, and can also glycosylate appropriate aglycone substrates. Earlier work in our lab (Pfeifer, et l. Science 291, 1790, 2001) has demonstrated that engineered derivatives of E. coli BL21 can also produce aglycones, and that the optimal temperature for polyketide production is similar to that reported above for TDP-desosamine biosynthesis and transfer. Therefore, together the two technologies could be used to produce biologically active erythromycins in E. coli. as illustrated in Example 4. Similar approaches could be used to engineer other deoxysugar biosynthetic and/or transfer pathways into E. coli, including the biosynthesis of cladinose, mycaminose, oleandrose, forosamine, daunosamine, mycarose, ascarylose, rhamnose, or mycosamine under conditions wherein the nucleotide diphosphate sugar is produced and the 6-deoxyglycosyltransferase is expressed. Such sugars are valuable metabolites in their own right. Moreover, since several other commercially important antibiotics such as tylosin, midecamycin, avermectin and candicidin also require glycosylation, our technology should find wide applications in the production and biosynthetic modification a variety of PK's in E. coli. Finally, the use of E. coli as a host should greatly facilitate the engineering of polyketide and deoxysugar pathways even further. For example, a high-throughput strain improvement program could be set up on a genetically engineered PKS using an antibiotic assay for biological function (as opposed to an analytical assay for chemical structure). Alternatively, by introducing the aglycone pathway into one strain of E. coli and the deoxysugar pathway into another, it should be possible to set up a secretor-converter experiment on petri-dishes that facilitates selection of mutant secretor strains which produce new PK's capable of killing the converter strain. Finally, if clinically relevant antibiotic resistance mechanisms are introduced in the converter host, such directed evolution experiments could also be used to discover new antibiotics that are active against resistant pathogens.

### EXAMPLE 4

### Production of 3-O-α-mycarosyl-erythronolide B in E. coli

Genes involved in the biosynthesis of mycarose are individually amplified by PCR using Deep Vent DNA polymerase (NEB) from chromosomal DNA of a mycarose-producing organism. Sources for mycarose biosynthetic genes include, for example, the tylC genes of Streptomyces fradiae (tylCII-tylCVII) together with the tylAI and tylAII genes (described in N. Bate et al., “The mycarose-biosynthetic genes of Streptomyces fradiae, producer of tylosin,” Microbiology (2000) 146, 139-146). Suitable mycarosyltransferase genes are available, for example, from Saccharopolyspora erythraea (eryCV) or other organisms.

Each pair of PCR primers is designed to introduce an NdeI site at the 5′ end and a SpeI site at the 3′ end of the gene amplified. PCR products are cloned into pCR-Blunt II-TOPO vector and the resulting plasmids are used to transform E. coli DH5α. The plasmids are digested with the enzymes NdeI and SpeI and fragments corresponding to each gene are cloned into a modified pET-24b (the modification consists of replacing the region between the XbaI and EcoRI sites in the multiple cloning cassette with the sequence 5′-TCTAGAAGGAGATATACATATGTGAACTAGTGAATTC-3′) previously digested with the same enzymes.

Individual mycarose biosynthetic genes are assembled into a synthetic operon as follows. A vector containing one gene of the synthetic operon is digested with the enzymes XbaI and SpeI, and the resulting mycarose gene-containing fragment is ligated to the vector containing a second gene digested with the enzyme SpeI. Plasmids harboring the two genes in the same orientation (as determined by restriction mapping) are selected and digested with SpeI, and ligated to the mycarose gene-containing fragment from a third mycarose gene-containing vector digested with the enzymes XbaI and SpeI. Plasmids harboring the three genes in the same orientation (as determined by restriction mapping) are selected, and the cycle is repeated until all required mycarose biosynthetic genes are assembled into the synthetic operon. Genes for the mycarosyltransferase (tylCV) and the 6-deoxyerythronolide B 6-hydroxylase (eryF) are added in similar fashion.

The resulting vector is used to transform the E. coli strain BL21 Codon Plus (Stratagene). Individual transformants are used to inoculate 15 ml Luria-Bertani cultures containing 50 μg/ml kanamycin and 0.5 μg/ml of 6-deoxyerythronolide B and are grown at 37° C. to A600 0.5-0.8 before the addition of IPTG to a final concentration of 0.5 mM. The cultures are then grown at 25° C. for 40 h and centrifuged. The supernatants are extracted with an equal volume of ethyl acetate, and the organic layer is dried over Na₂SO4, evaporated to dryness, and redissolved in ethanol. The presence of mycarosyl-EB is confirmed by LC/MS ([M+H]+m/z 547).

The above experiments have shown that E. coli can successfully synthesize TDP-desosamine, and can also glycosylate appopriate aglycone substrates. Earlier work in our lab (Pfeifer, et 0.1. Science 291, 1790, 2001) has demonstrated that engineered derivatives of E. coli BL21 can also produce aglycones, and that the optimal temperature for polyketide production is similar to that reported above for TDP-desosamine biosynthesis and transfer. Therefore, together the two technologies could be used to produce biologically active erythromycins in E. coli. as illustrated in Example 4. Similar approaches could be used to engineer other deoxysugar biosynthetic and/or transfer pathways into E. coli, including the biosynthesis of cladinose, mycaminose, oleandrose, forosamine, daunosamine, mycarose, ascarylose, rhamnose, or mycosamine under conditions wherein the nucleotide diphosphate sugar is produced and the 6-deoxyglycosyltransferase is expressed. Such sugars are valuable metabolites in their own right. Moreover, since several other commercially important antibiotics such as tylosin, midecamycin, avermectin and candicidin also require glycosylation, our technology should find wide applications in the production and biosynthetic modification a variety of PK's in E. coli. Finally, the use of E. coli as a host should greatly facilitate the engineering of polyketide and deoxysugar pathways even further. For example, a high-throughput strain improvement program could be set up on a genetically engineered PKS using an antibiotic assay for biological function (as opposed to an analytical assay for chemical structure). Alternatively, by introducing the aglycone pathway into one strain of E. coli and the deoxysugar pathway into another, it should be possible to set up a secretor-converter experiment on petri-dishes that facilitates selection of mutant secretor strains which produce new PK's capable of killing the converter strain. Finally, if clinically relevant antibiotic resistance mechanisms are introduced in the converter host, such directed evolution experiments could also be used to discover new antibiotics that are active against resistant pathogens.

### EXAMPLE 5

### Preparation of Erythromycins in E. coli

A strain of E. coli producing erythromycins is constructed as follows. Suitable host strains include E. coli cells expressing one or more genes conferring erythromycin resistance, for example the strain E. coli BM2570, which expresses ermBC as described in Brisson-Noel et al., “Evidence for natural gene transfer from gram-positive cocci to Escherichia coli,” J Bacteriology (1988) 170(4): 1739-45. The final strain will comprise genes for the 6-deoxyerythronolide B polyketide synthase, or variant thereof, along with genes encoding the biosynthesis and transfer of L-mycarose and D-desosamine, the genes encoding the C-6 and C-12 hydroxylases and the 3″-O-methyltransferase. The final strain will also comprise genes for the biosynthesis of an appropriate starter unit and the required methylmalonyl-CoA extender units as described in PCT publications WO 01/27306 and WO 01/31049, which are incorporated herein by reference. Techniques for introducing the genes for biosynthesis and transfer of L-mycarose and D-desosamine are described in the Examples above. Techniques for introducing the polyketide synthase genes are described in PCT publication WO 01/31035, which is incorporated herein by reference. Techniques for producing mutated forms of the polyketide synthases, for example by domain exchange to alter the selectivity of acyltransferases or β-keto-modifying domains, are provided in U.S. Pat. Nos. 6,391,594; 6,403,775; and 6,399,789, each of which is incorporated herein by reference. Techniques for introducing the genes for the C-6 and C-12 hydroxylases are as described in the Examples above. Techniques for expressing the gene for the 3″-O-methyltransferase are described in Paulus et al., “Mutation and cloning of eryG, the structural gene for erythromycin O-methyltransferase from Saccharopolyspora erythraea, and expression of eryG in Escherichia coli,” J. Bacteriology (1990) 172(5): 2541-6. Suitably modified erythromycin derivatives may be obtained by appropriate selection of the genes in the producing host; for example, 6-deoxyerythromycins may be produced by exclusion of the 6-hydroxylase gene. The introduced biosynthetic genes are typically put under the control of inducible promoters, such as the lac promoter that is induced by addition of isopropyl-β-D-thiogalactopyranoside (IPTG).

To produce erythromycins using these host cells, cultures of the cells are grown in an appropriate medium, for example Luria-Bertani broth, at temperatures of 30-40° C., preferably 37° C., until the cells reach a density suitable for induction of expression of the biosynthetic genes. Typically, this cell density is about 1.0 optical density unit as measured by light scattering at 600 nm. At this point, the culture is chilled to 20° C., and the inducing agent, for example IPTG, is added. The culture is allowed to grow at this lower temperature, and aliquots are periodically removed and assayed for erythromycin production. Suitable assays include, for example, HPLC-based assays using erythromycin standards and detection by evaporative light scattering or mass spectrometry, or biological assays such as antimicrobial activity against a suitable test strain, for example Micrococcus luteus. When the rate of erythromycin production is observed to level off, the culture is harvested by centrifugation. The supernatant is adjusted to pH 9 and extracted with an organic solvent such as dichloromethane or ethyl acetate. The organic extract is dried, for example over sodium sulfate, filtered, and evaporated to provide the crude erythromycin. Purified erythromycin can be obtained using procedures known in the art, for example chromatography or crystallization.

What is claimed is:

1. A recombinant E. coli host cell containing an expression system for producing at least one nucleotide diphosphate 6-deoxy-sugar.

2. The host cell of claim 1, further comprising an expression system for expressing 6-deoxyglycosyl transferase.

3. The host cell of claim 2, further comprising an expression system for the synthesis of a polyketide.

4. The host cell of claim 1, wherein said sugar is selected from the group consisting of desosamine, cladinose, mycaminose, oleandrose, forosamine, daunosamine, mycarose, ascarylose, rhamnose, and mycosamine under conditions wherein the nucleotide diphosphate sugar is produced and the 6-deoxyglycosyltransferase is expressed.

5. The host cell of claim 4, wherein said sugar is D-desosamine.

6. The host cell of claim 1, wherein the expression system comprises desosamine biosynthetic genes from Streptomyces venezuelae, Saccharopolyspora erythraea, Streptomyces narbonesis, or Streptomyces antibioticus.

7. The host cell of claim 6, wherein said desosamine biosynthetic genes are from Streptomyces venezuelae.

8. The host cell of claim 7, wherein the desosamine biosynthetic genes comprise desi-des VI and des VIII genes.

9. The host cell of claim 8, further comprising an expression system for expressing a desosaminyltransferase.

10. The host cell of claim 3, wherein the expression system for the synthesis of a polyketide comprises genes encoding a 6-deoxyerythronolide B synthase.

11. The host cell of claim 10, further comprising an expression system for a 6-erythronolide B 6-hydroxylase.

12. The host cell of claim 11, wherein the expression system for producing at least one nucleotide diphosphate 6-deoxy-sugar comprises genes encoding enzymes that produce TDP-mycarose, and wherein the expression system for expressing a 6-deoxyglycosyltransferase expresses a mycarosyltransferase.

13. The host cell of claim 11, further modified with an expression system for an erm ribosomal methyltransferase.

14. The host cell of claim 13, further comprising an expression system for producing TDP-desosamine and a desosaminyltransferase.

15. The host cell of claim 14, further comprising an expression system for an erythromycin D 12-hydroxylase.

16. The host cell of claim 15, further comprising an expression system for an erythromycin C 3″-O-methyltransferase.

17. A method for producing a glycosylated polyketide comprising feeding a polyketide to a culture of the host cells of claim 2 under conditions wherein the nucleotide diphosphate 6-deoxy-sugar is produced and the 6-deoxyglycosyltransferase is expressed.

18. The method of claim 17, wherein the polyketide is a 6-deoxyerythronolide B.

19. A method of producing an erythromycin analog comprising culturing the host cells of claim 16 under conditions wherein the genes in each expression system are expressed to produce functional enzymes.

20. The host cell of claim 1 wherein the expression system for producing nucleotide diphosphate 6-deoxy-sugar does not comprise biosynthetic genes from Micromonospora megalomicea.


SEQUENCE LISTING (1 sequence)

SEQ ID NO: 1 - DNA, 37 bp, Artificial Sequence
  modified pET-24b
tctagaaggagatatacatatgtgaactagtgaattc